(19) United States
(12) Patent Application Publication
(10) Pub. No.: US 2010/0326280 A1
(43) Pub. Date: Dec. 30, 2010

(54) CAR PLUG-IN SCENTED AIR FRESHENER

(76) Inventor: Sharon Hicks, Henderson, NV (US)

Correspondence Address: ROBERT J. SAYFIE, P.C., 161 OTTAWA AVENUE NW, SUITE 407, GRAND RAPIDS, MI, 49503, US

(21) Appl. No.: 12/494,354

(22) Filed: Jun. 30, 2009

(51) Int. Cl. A61L 9/00

(52) U.S. Cl. 96/222

(57) ABSTRACT

An electrically powered air freshener for motor vehicles. The air freshener has containers that contain an air freshener substance that can evaporate from the container, and can be displaced from the air freshener via a fan. The motor and fan of the air freshener may be turned on, off, or the speed of the fan can be controlled.

### GOVERNMENT RIGHTS

This invention was not made with Government support under. The Government does not have any rights in this invention.

### BACKGROUND OF THE INVENTION

The present invention is an electric, refillable air fresher for automobiles. More particularly, an electric air freshener that is capable of being plugged into a power source of a motor vehicle.

Currently, air fresheners for vehicles do for motor vehicles do not have the combination of being powered with a fan and refillable.

Presently motor vehicle users can use a hanging card that hangs from the rear-view mirror, or a pump or spray or aerosol, or a vent-attached system.

The hanging card type of air freshener may create a visual obstruction to drivers and is therefore potentially dangerous.

A pump or aerosol may be dangerous to keep in a hot motor vehicle interior, as the pressurized container may become over pressurized, and possibly leak or explode, or otherwise become damaged. In addition, these liquids or gasses may damage the interior.

A vent-attached air freshener system requires use of the vehicle air conditioner or heat system, which may not be desirable by the user or it may not operate properly. Also, because a vent-attaching air freshener may require the use of the dashboard vent to receive needed air contact, may users of that product type are only left coated directly with those scents, or must choose to not that the air current directed in their vicinity in order to avoid that personal perfuming.

The prior art air freshener may also be set on a dashboard, which, like the hanging-type may pose an eyesore or a visual obstruction.

As can be seen, there is a need for a motor vehicle air freshener that can be refillable and powered with a fan. There is also a need for an air freshener that is not reliant upon a dashboard vent, as the user may want to use only the floor vent, or vice-versa.

There is also a need for an air freshener that is controllable as to its power and whether the user wants it turned on or off.

There is also a need for an air freshener that can be refilled with a new scented cartridge or container when desired.

There is also a need to an air freshener that can appeal to many consumers who often times use their vehicles as their work offices such as real estate professionals, contractors, outdoor advertisers and public work officials.

There is also a need for an air freshener that can be made in various sizes and shapes to fit within automobiles.

### SUMMARY OF THE INVENTION

One aspect of the present invention is a An air freshener (10), comprising: a housing (30); said housing (30) having a vent (40) to release an air freshener substance from a container (70) to through said vent (40); a fan (90) operatively connected to a motor (110); a means to operate said motor (50); and a container (70) removably attached to said housing (30), whereby the container has an opening (72) so that an air freshener substance may evaporate from said container (70).

These and other features, aspects and advantages of the present invention will become better understood with reference to the following drawings, description and claims.

### BRIEF DESCRIPTION OF THE DRAWINGS

FIG. 1 is a pictorial of an embodiment of the present invention;

FIG. 2 is a second pictorial of an embodiment of the present invention; and

FIG. 3 is a third pictorial of an embodiment of the present invention.

FIG. 4 is a pictorial of an embodiment of the present invention with the removable container removed from the housing;

FIG. 5 is a cut-away view of an embodiment of the present invention;

FIG. 6 is an embodiment of the present invention illustrating the air flow; and

FIG. 7 is a pictorial of the present invention is use in a car.

### DETAILED DESCRIPTION OF THE INVENTION

### Reference Numerals

10 air freshener, plug in air freshener, powered motor vehicle air freshener
20 power receiver
30 housing
32 front side
34 rear side
40 vent
50 means to operate said motor or dial
60 indicator
62 extension
64 receiver
70 container or cartridge
72 container opening
80 rear opening
90 fan
100 air flow
110 motor
120 cartridge host
130 light

The following detailed description is of the best currently contemplated modes of carrying out the invention. The description is not to be taken in a limiting sense, but is made merely for illustrating the general principles of the invention, since the scope of the invention is best defined by the appended claims.

The present invention 10 may be referred to herein as a powered motor vehicle air freshener 10, air freshener 10, or a plug in scented air freshener 10.

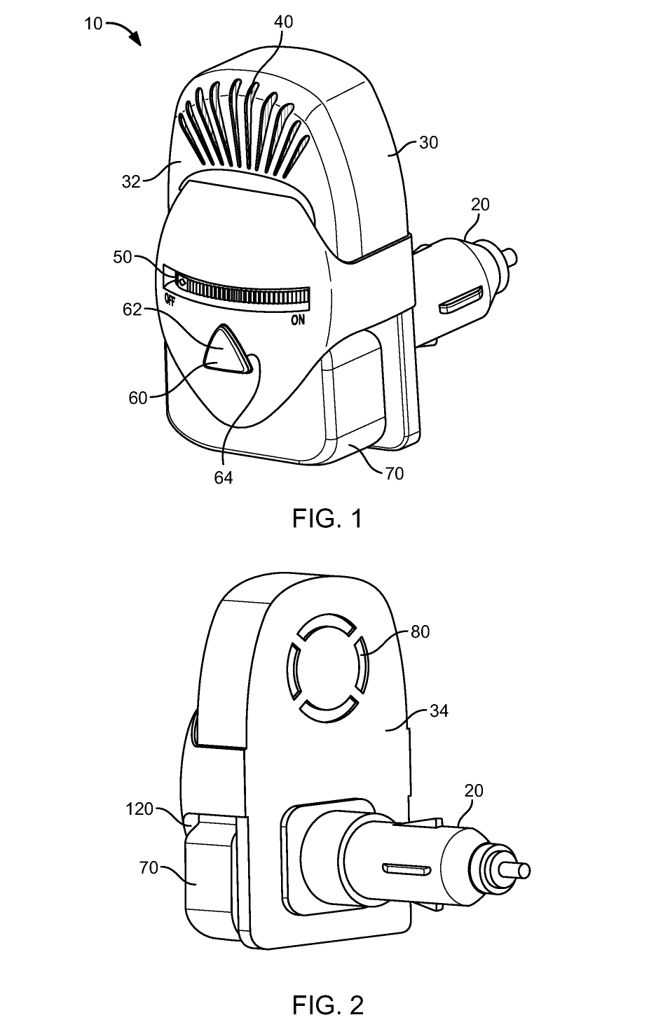

FIG. 1 illustrates an embodiment of the present invention showing a powered motor vehicle air freshener 10. The air freshener 10 may have a housing 30 that has a vent 40 to dissipate an air freshener that may be contained in a removable container 70. The air freshener may be a liquid or gelatin material that may be located in the container 40. The air freshener 10 may be capable of being plugged into a power outlet of a motor vehicle such as a cigarette lighter, or a standard two-pronged plug (not shown).

The housing 30 may be made of various plastic materials of adequate durability and tolerance, such as polypropylene (“PP”) or polyethylene terephthalate (“PET”).

The cartridge 70, container 70, removable container 70, or removable cartridge 70 may be made of various plastic materials of adequate durability and tolerance, such as high density polyethylene (“HDPE”), expanded polystyrene (“PS”) or polyester resin.

The cartridge 70 may be inserted within the host 30 or housing 30 from the top or bottom.

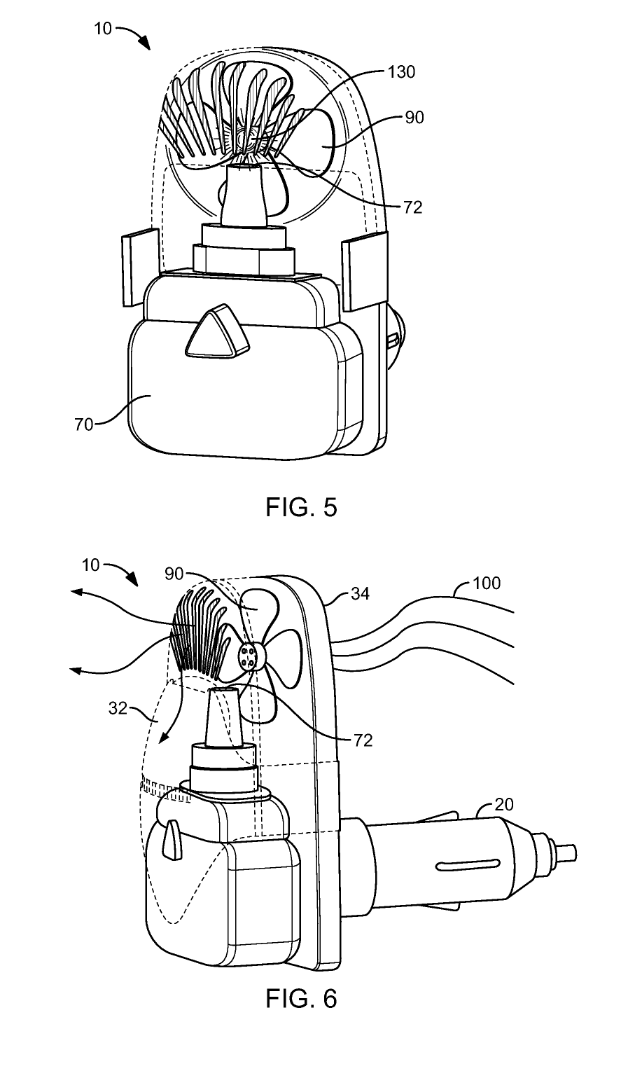

FIG. 1 also shows a power receiver 20 that may be plugged into a motor vehicle power source. The power receiver 20 may extend rearwardly from the housing 30. A means to operate said 50 or a dial 50 may be accessible on the front side 32. The means to operate the motor 50 may be operably connected to a motor 110 (not shown) to control the operation of a fan 90 illustrated in FIGS. 5 and 6. The means to operate the motor 50 may be capable of turning the motor and fan, on, off, and to vary the speed of the motor and fan. In one embodiment, the means to operate the motor 50 may be a dial 50. The motor 110 may receive its energy from electricity through the power receiver 20. The dial 50 may be able to control the motor 110 by turning the motor 110 off and on, and controlling the speed of the motor 110.

FIG. 2 illustrates that the housing 30 may have a cartridge host 120, which is an open area that may receive a container 70 or cartridge 70. The container 70 may have an extension 62 that may removably engage with a receiver 64 of the housing. The receiver 64 may be an open-area or cut-out in the front side 32. The receiver 64 may be able to receive the extension 62 of comparable shape and size, so that if the front side 32 is pulled forwardly, or if the extension 62 is displaced or pushed rearwardly, then the cartridge or container 70 may be removed from the housing 30. In one embodiment the housing 30 is made of plastic that is flexible enough to bend so that the extension 62 may be removable engaged with the receiver 64.

The container 70 or cartridge 70 may contain air freshener's with different scents, such as pine, cinnamon, pineapple, and other scents. The container 70 or cartridge 70 may also offer scents with aromatherapy benefits.

FIG. 2 illustrates the rear side 34 may have a rear opening 80 to allow air to enter the housing 30 so that air may be displaced from the vent 40. In one embodiment of the present invention 10, the air may adopt an air flow path 100 as illustrated in FIG. 6. FIG. 2 also illustrates the cartridge host 120 adaptable to receive a cartridge 70 or container 70.

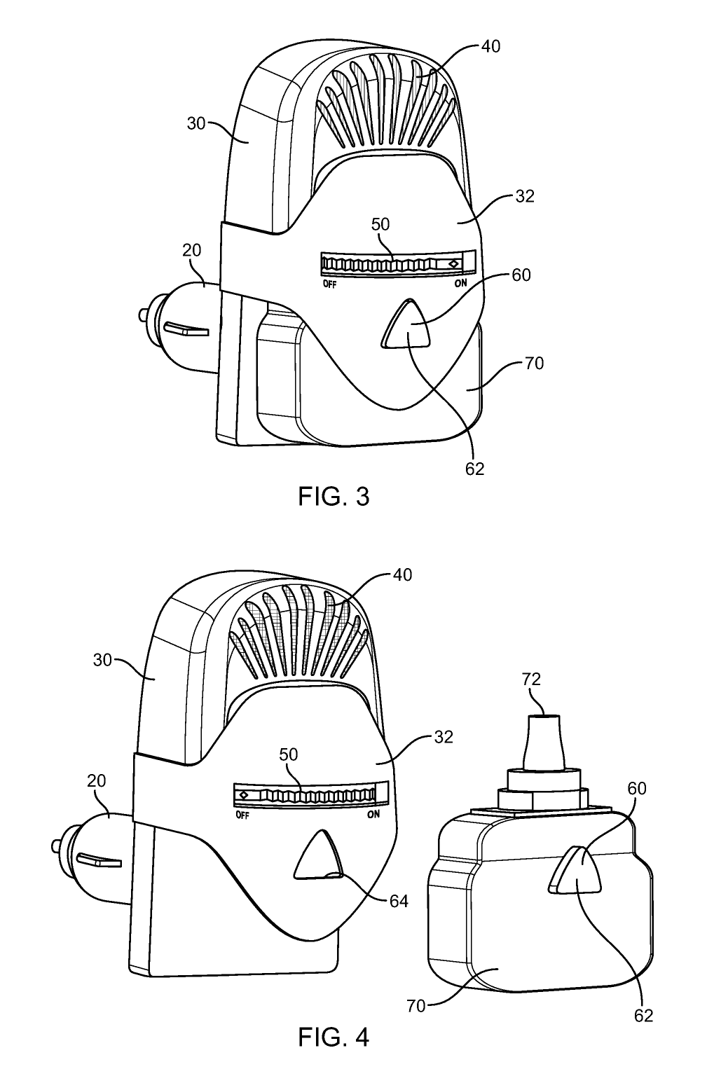

FIG. 3 illustrates substantially another view similar to FIG. 1.

FIG. 4 illustrates what one embodiment of the container 70 or cartridge 70 may look like. If the container 70 is transparent or translucent, then the extension 62 may act as a level indicator or indicator 60 of the air freshener substance. The receiver 64 is illustrated as being adaptable to receive the extension 62. In one embodiment the receiver 64 and extension 62 may be of a triangular shape. FIG. 4 also illustrates a container opening 72, which enable the air freshener substance within the container 70 to evaporate from said container 70. The container opening 72 is also illustrated in FIGS. 5 and 6.

FIG. 5 illustrates a sectional front view showing the fan 90 and light 130.

FIG. 6 illustrates one example of the path of air flow 100 in the present invention 10, when the air flows from the rear opening 80, by the blades of the fan 90, and out through the vent 40.

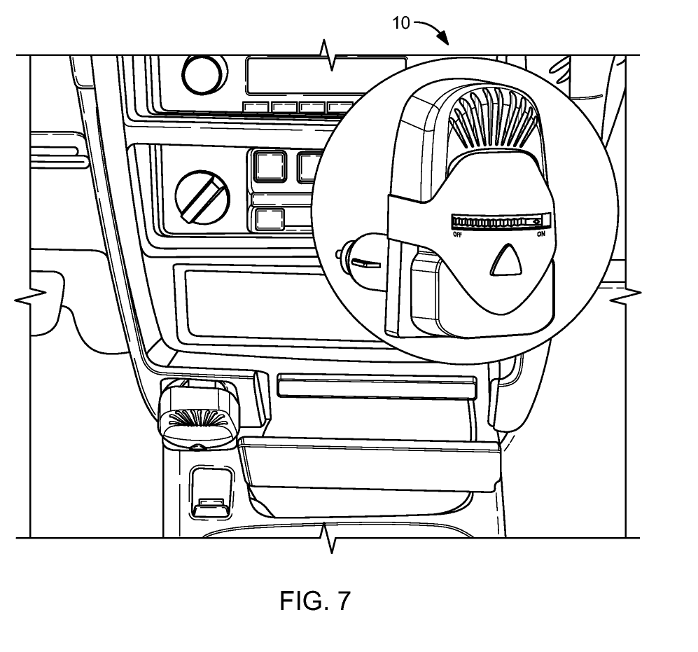

FIG. 7 illustrates a motor vehicle interior in which the air freshener 10 may be used.

The present invention 10 may refresh the environment of motor vehicle interiors. The present invention 10 may provide needed odor control within the motor vehicle as the vehicle may produce and travel through odorous gasses.

The fan 90 may be turned on or off, and the fan speed may be controlled via the dial 50. The present invention 10 may have a light 130 (illustrated in FIG. 5) that may indicate the status of use. For example, the light 130 may turn on when the air freshener 10 is on, and the light 130 may turn off when the air freshener 10 is turned off.

The present invention air freshener 10 may provide a pleasant scent throughout the interior of an automobile, and in a format that is better, safer, more consistent and more controllable than all other formats of motor vehicle air freshener.

In one embodiment the housing 30 may be made of HDPE and in approximate two-and-one-half-inch height by two-inch width by one-inch depth (2½″×2″×1″). The top corners of the housing 30 may be rounded.

In one embodiment of the present invention 10, fixed within the interior top-rear of the housing 30 may be a fan 90 that may have a total blade span of about 1 inch. Below and connected by wire to the fan 90 may be a motor 110 for the fan 90. The motor 110 may be connected by wire to a twelve-volt adaptor module or power receiver 20 that may extend substantially rearwardly from the housing 30 and oriented substantially perpendicular with respect to the rear side.

In one embodiment the bottom-interior of the housing 30 may be hollowed, and for acceptance of a cartridge 70 or container 70 that holds the air freshener substance, which may be a liquid or a gel, or other air freshener substance.

In one embodiment the front side 32 may have a rotating dial 50, and a light emitting diode (not shown).

In one embodiment the container 70 removably snaps into the housing 30.

It should be understood, of course, that the foregoing relates to exemplary embodiments of the invention and that modifications may be made without departing from the spirit and scope of the invention as set forth in the following claims.

I claim:

1. An air freshener (10), comprising:
a housing (30);
said housing (30) having a vent (40) to release an air freshener substance from a container (70) to through said vent (40);
a fan (90) operatively connected to a motor (110);
a means to operate said motor (50); and
a container (70) removably attached to said housing (30), whereby the container has a container opening (72) so that an air freshener substance may evaporate from said container (70).

2. The apparatus of claim 1, further comprising:
said housing (30) having a rear side (34), and said rear side having a rear opening (80) whereby an air flow (100) can flow from said rear opening (80) through said vent (40).

3. The apparatus of claim 1, further comprising:
a light (130) disposed inside said housing (30) that turns on when said fan (90) is on, and said light (130) turns off when said fan (90) is off.

4. The apparatus of claim 1, further comprising:
a power receiver (20) to receive electrical power and supply electrical power to said motor (50.

5. The apparatus of claim 1, wherein said means to operate said motor (50) is a dial (50) that can turn the motor (100) off, on, and vary the speed of the motor (110).

6. The apparatus of claim 1, wherein said housing (30) has a front side (32) and said vent (40) is disposed on said front side (32).

7. The apparatus of claim 1, further comprising:
said housing (30) having a cartridge host (120), said housing also having a receiver (64), and said container (70) having an extension (62) that is removably engaged with said receiver (64) so that the container (70) can retained in position within said cartridge host (120) and said container (70) can be removed from said cartridge host (120).

8. The apparatus of claim 1, wherein said container (70) is at least one of either transparent or translucent so that said extension (62) can be viewed when said container (70) is disposed within said cartridge host (120) and then if there is air freshener substance within said container (70) at the level of the extension (62), then it can be viewed through the indicator (60).
